(19) United States
(12) Patent Application Publication
(10) Pub. No.: US 2011/0015619 A1
(43) Pub. Date: Jan. 20, 2011

(54) WOUND DRESSINGS FOR NEGATIVE PRESSURE THERAPY IN DEEP WOUNDS AND METHOD OF USING

(76) Inventors: Pal Svedman, Malmo (SE); David M. Turney, Germantown, MD (US); Richard C. Vogel, Gaithersburg, MD (US); Mark S. Meents, Germantown, MD (US)

Correspondence Address: A PATENT LAWYER CORP, PLC;R WILLIAM GRAHAM, 22 S ST CLAIR ST, DAYTON, OH, 45402, US

(21) Appl. No.: 12/504,438

(22) Filed: Jul. 16, 2009

(51) Int. Cl. A61M 27/00; A61K 47/32

(52) U.S. Cl. 604/543; 514/772.3

(57) ABSTRACT

A method for providing negative pressure wound healing in a deep wound having a cavity includes the steps of (a) fashioning at least a first part of a therapeutic dressing to fit generally within the cavity, (b) operably associating a fluid moving means with the therapeutic dressing for enabling one of compressing the therapeutic dressing and transferring fluid across at least part of the therapeutic dressing, and (c) enabling the fluid moving means in a manner to deliver therapy to the patient. A therapeutic member for use in negative pressure wound healing includes a porous member formed and includes a hydrogel coating on a side thereof.

### BACKGROUND

1. Field of Invention

This invention is generally directed to a therapeutic method and device for the promotion of wound healing. More particularly, the invention relates to a method and device for providing negative pressure wound healing in deep wounds with tunnels, fistulas, grafts or external fixation devices.

2. Related Art

Negative pressure wound therapy (NPWT), also known as vacuum drainage or closed-suction drainage, is known. A vacuum source is connected to a semi-occluded or occluded therapeutic member, such as a compressible wound dressing. Various porous dressings comprising gauze, felts, foams, beads and/or fibers can be used in conjunction with an occlusive semi-permeable cover adhering to the skin around the wound and a controlled vacuum source. In addition to negative pressure, there exist pump devices configured to supply positive pressure to another therapeutic member, such as an inflatable cuff, used for hemostasis and various other medical therapies.

In addition to using negative pressure wound therapy, many devices employ concomitant wound irrigation. For example, a known wound healing apparatus includes a porous dressing made of polyurethane foam placed adjacent a wound and covered by a semi-permeable and flexible plastic sheet. The dressing further includes fluid supply and fluid drainage connections in communication with the cavity formed by the cover, foam, wound surface and skin. The fluid supply is connected to a fluid source that can include an aqueous topical anesthetic or antibiotic solution, isotonic saline, or other medicaments for use in providing therapy to the wound. The fluid drainage can be connected to a vacuum source where fluid can be removed from the cavity and subatmospheric pressures can be maintained inside the cavity. The wound irrigation apparatus, although able to provide efficacious therapy to wounds which are substantially planar, requires adaptation to treat wounds with tunnels, fistulas, grafts or external fixation devices. Such a device using substantially planar dressings does not address various factors concerning patients with deep wounds whose surface is not planar but curvilinear, such as a tunnel or fistula.

Some devices use vacuum sealing of wound dressings consisting of polyvinyl alcohol foam cut to size and stapled to the margins of the wound. Such dressings are covered by a semi-permeable membrane while suction and fluid connections are provided by small plastic tubes are introduced into the foam and patient's skin. Currently, such devices alternate in time between vacuum drainage and the introduction of aqueous medicaments to the wound site, but do not do both simultaneously. Also, current devices in the marketplace provide therapy using vacuum to apply negative pressure for a period of time (a “pressure application” mode) and then release vacuum and negative pressure (a so-called “relaxation” mode). While the prior devices provide useful therapy, there remains a need to improve on the devices and methods of applying negative pressure wound therapy in deep wounds or wounds with external fixation devices.

### SUMMARY OF THE INVENTION

It is an object to improve wound healing.

It is another object to improve devices for use in treating wounds.

It is an object to improve methods for treating wounds.

It is yet another object to improve wound therapy by adapting the wound dressing for deep wounds with tunnels, fistulas, grafts or external fixation devices.

It is yet another object to provide a therapeutic device for treating wounds which is equipped for predetermined therapy parameters of time and pressure.

The invention is directed to a method and device for providing negative pressure wound healing in deep wounds with tunnels, fistulas, grafts or external fixation devices. A method for providing negative pressure wound healing in a deep wound having a cavity includes the steps of (a) fashioning at least a first part of a therapeutic dressing to fit generally within the cavity; (b) operably associating a fluid moving means with the therapeutic dressing for enabling one of compressing the therapeutic dressing and transferring fluid across at least part of the therapeutic dressing; and (c) enabling the fluid moving means in a manner to deliver therapy to the patient.

The therapeutic dressing in step (a) is further characterized to include a first foam portion which is cut to generally fit within the cavity and a second foam portion overlying and extending to but not beyond the wound margins and includes a semi-permeable membrane wrapped about the foam portions and in generally sealed contact with skin about the wound.

The step (c) can be such to enable the fluid moving means for a period of time to foster at least partial closure of the wound rendering a least a partially closed cavity and cause at least partial migration the first part of the therapeutic dressing from the wound cavity and further includes the steps of (d) removing at least part of the first part of the therapeutic dressing; (e) providing a therapeutic dressing having a first part with reduced size relative the first part of the therapeutic dressing in step (a) to fit generally within the at least partially closed cavity; (f) operably associating a fluid moving means with the therapeutic dressing with reduced size for enabling one of compressing the therapeutic dressing and transferring fluid across at least part of the therapeutic dressing; and (g) enabling the fluid moving means in a manner to deliver therapy to the patient.

The step (g) can be such to enable the fluid moving means for a period of time to foster closure of the wound rendering a closure of the cavity and cause migration of the first part of the therapeutic dressing from the wound cavity and further includes the steps of (h) removing the first part of the therapeutic dressing; (i) providing a therapeutic dressing having a second compressible part over the closed cavity extending to but not beyond the wound margins; (j) operably associating a fluid moving means with the therapeutic dressing for enabling one of compressing the therapeutic dressing and transferring fluid across at least part of the therapeutic dressing; and (k) enabling the fluid moving means in a manner to deliver therapy to the patient. The step (k) can be such to enable the fluid moving means for a period of time to render closure of the wound and further includes the step of (l) removing the therapeutic dressing.

Another embodiment is directed to a method for providing negative pressure wound healing in a wound having a cavity in which part of an external fixation device extends, which includes the steps of (a) fashioning at least a first part of a therapeutic dressing to fit generally about the part of the external fixation device adjacent the cavity of the wound, (b) operably associating a fluid moving means with the therapeutic dressing for enabling one of compressing the therapeutic dressing and transferring fluid across at least part of the therapeutic dressing, and (c) enabling the fluid moving means in a manner to deliver therapy to the patient.

The therapeutic dressing in step (a) is further characterized to include a porous member formed to fit generally about the part of the external fixation device and includes a hydrogel coating on a side thereof. The hydrogel includes polypropylene glycol, isophorone diisocyanate, and polyethylene oxide-based diamine in an aqueous solution of NaCl.

Control means can also preferably control operation of the device in a manner to restrict use of the fluid moving means by the patient in accordance with a predetermined treatment plan or duration and render the pump inoperable. The power source can be a rechargeable power source.

More particularly, a wound irrigation system can use a fluid moving means, such as a diaphragm or piston-type pump, to raise, compress and transfer fluid in an electromechanical vacuum apparatus which includes a control means, such as a microprocessor-based device, having stored thereon software configured to control the electromechanical vacuum apparatus, and including one of a timer, means for remote control of the system, and means to restrict the operation of the apparatus to a predetermined treatment plan or duration.

A first vacuum pump can be electrically associated with the microcontroller and capable of generating a vacuum. An optional second vacuum pump is electrically associated with the microcontroller and is capable of maintaining a predetermined vacuum level. A first electronic vacuum-pressure sensor can be operably associated with the vacuum pump(s) and the microcontroller for monitoring vacuum level.

A fluid-tight collection canister can be provided and can include an integrated barrier, such as a hydrophobic filter, to prevent contents from escaping the canister. Single-lumen tubing can be associated with the canister and vacuum pump(s) for communicating vacuum pressure therefrom. A second electronic vacuum-pressure sensor can be operably associated with the canister and the microcontroller for monitoring canister vacuum.

A dressing includes a porous material and semi-permeable flexible cover. Single-lumen tubing is associated with the dressing and the canister to communicate vacuum pressure therefrom. The deep wound porous dressing is configured as a cone, cylinder, wedge, pyramid or a self-sealing disk with an aperture and a radial slit to accommodate external fixation devices such as pins.

The electromechanical vacuum apparatus housing may incorporate a compartment that can hold the irrigation vessel. The electromechanical vacuum apparatus can preferably include a device for regulating the quantity of fluid flowing from said irrigation vessel to said dressing. This device can comprise a mechanical or pneumatically actuated valve or clamp.

The electromechanical vacuum apparatus may include commercially available disposable storage batteries enabling portable operation thereof. Alternative power sources include rechargeable or reprocessable batteries which are removably connected to a housing, which contains the fluid moving means and control means, both of which require power in a waterproof environment. Other alternative power sources are solar energy, a manually operated generator in combination with a storage device such as a supercapacitor, or a pneumatic accumulator.

An embodiment of the invention includes a method for improving the generation and control of a therapeutic vacuum. In this embodiment, a multi-modal algorithm monitors pressure signals from a first electronic vacuum-pressure sensor associated with a vacuum pump and capable of measuring the output pressure from the pump. The algorithm further monitors pressure signals from a second electronic vacuum-pressure sensor associated with a collection canister and capable of measuring the subatmospheric pressure inside the canister. The second electronic vacuum-pressure sensor may also be associated with the wound dressing and capable of measuring the subatmospheric pressure inside the dressing. The canister is connected to the vacuum pump by a single-lumen tube that communicates subatmospheric pressure therefrom. The canister is connected to a suitable dressing by a single-lumen tube that communicates subatmospheric pressure thereto.

At the start of therapy, both the first and second electronic vacuum-pressure sensors indicate the system is equilibrated at atmospheric pressure. A first-mode control algorithm is employed to rapidly remove the air in the canister and dressing, and thus create a vacuum. The first-mode implemented by the control algorithm is subsequently referred to herein as the “draw down” mode. Once the subatmospheric pressure in the canister and dressing have reached a preset threshold as indicated by the first and second electronic vacuum-pressure sensors respectively, the algorithm employs a second-mode that maintains the desired level of subatmospheric pressure in both the canister and the dressing for the duration of the therapy. The second-mode implemented by the control algorithm is subsequently referred to herein as the “maintenance” mode.

The second-mode control algorithm is configured to operate the vacuum pump at a reduced speed thus minimizing unwanted mechanical noise. In an alternative embodiment, a second vacuum pump can be used for the maintenance mode, which has a reduced capacity, is smaller, and produces significantly lower levels of unwanted mechanical noise. The second-mode control algorithm is configured to permit the maintenance of vacuum in the presence of small leaks, which invariably occur at the various system interfaces and connection points. The method can be performed by, for example, a microprocessor-based device.

The control means can be provided with a timer for restricting the use as a function of a predetermined time. Alternatively, an identification member can be provided with the device such that the control means restricts use as a function of the identification member. The control means may include a Radio Frequency Identification Chip (RFID) chip available under the trademark Omni-ID™. The control means can be operably associated with a remote control for restricting the use of the device.

### BRIEF DESCRIPTION OF THE DRAWINGS

FIG. 1 is a schematic illustrating the device of the invention.

FIG. 1A is a cross-sectional view of a deep wound extending below the skin.

FIG. 1B is another cross-sectional view of a deep wound extending below the skin.

FIG. 2A is a compressible foam dressing in the form of a frustum of a cone.

FIG. 2B is compressible foam dressing in the form of a wedge or pyramid.

FIG. 2C is a compressible foam dressing disk with an aperture through the center of the disk and a hydrogel coating on one surface for adherence to an external fixation device.

FIG. 3 is an external fixation device (EFD) with pins penetrating the apertures of the disks of FIG. 2C and extending into an underlying bony structure.

FIG. 4 is the compressible foam dressing of FIG. 2A trimmed at its narrow end to approximate the surfaces of the dressing with the deepest surfaces of a wound.

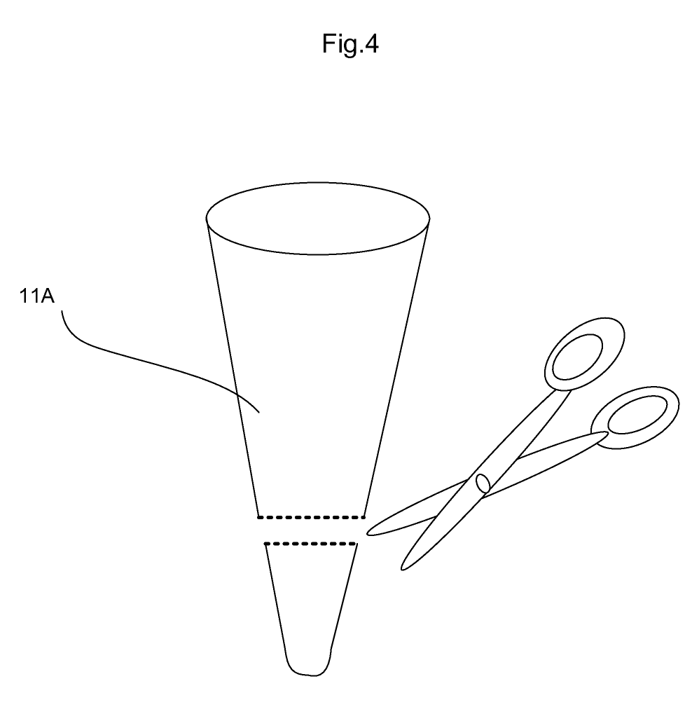

FIG. 5 is the trimmed compressible foam dressing of FIG. 4 placed into the wound and trimmed at its wide end to be substantially coplanar with the periwound area on the patient's skin.

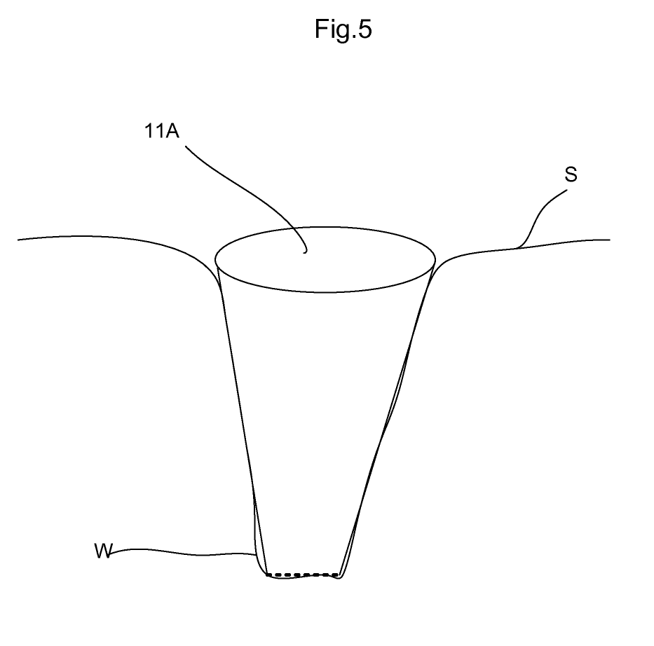

FIG. 6 is the trimmed compressible foam dressing of FIG. 5 in fluidic contact with a planar foam dressing, covered with a semi-permeable adhesive cover, which forms a fluidically sealed cavity with the patient's skin.

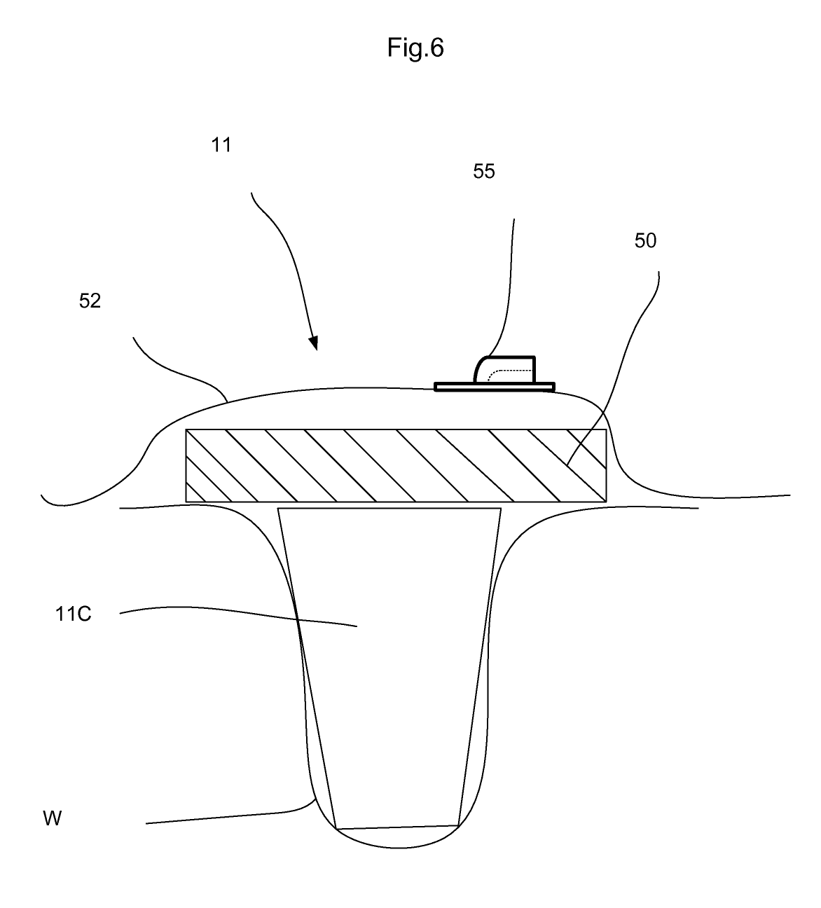

FIG. 7 is a replacement compressible foam dressing which has been trimmed to fit the decreased volume of the partially healed wound.

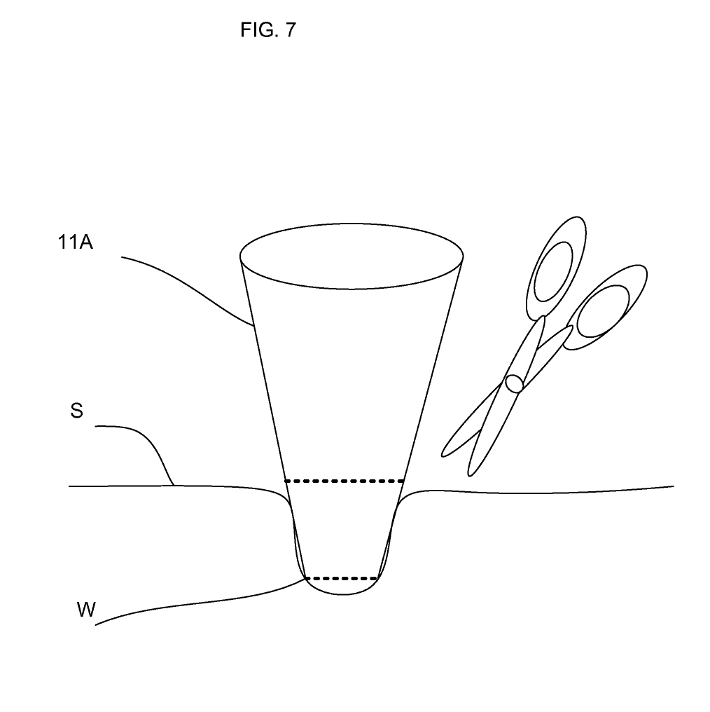

FIG. 8 is a further replacement of a compressible foam dressing which has been trimmed to fit the further decreased volume of the nearly healed wound.

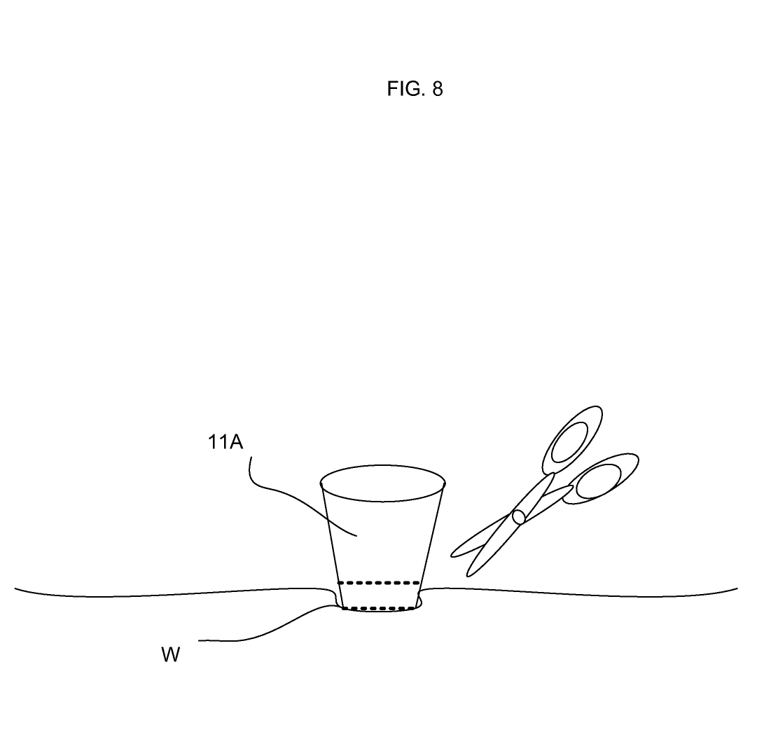

FIG. 9 is a healed wound after dressings and therapeutic devices are removed.

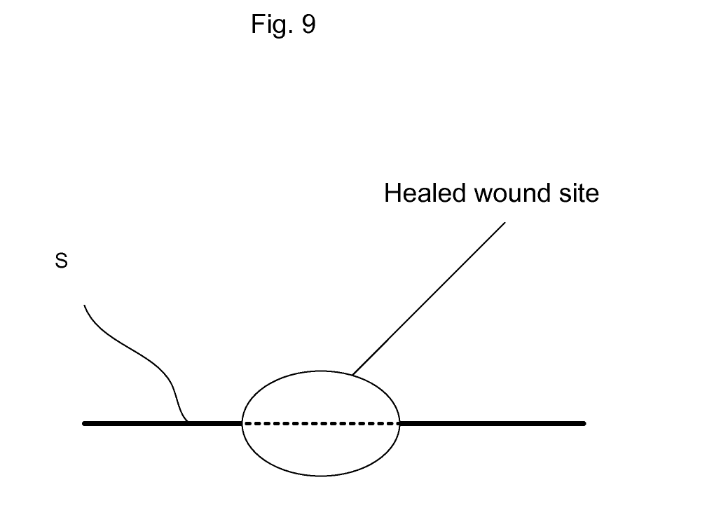

### DETAILED DESCRIPTION

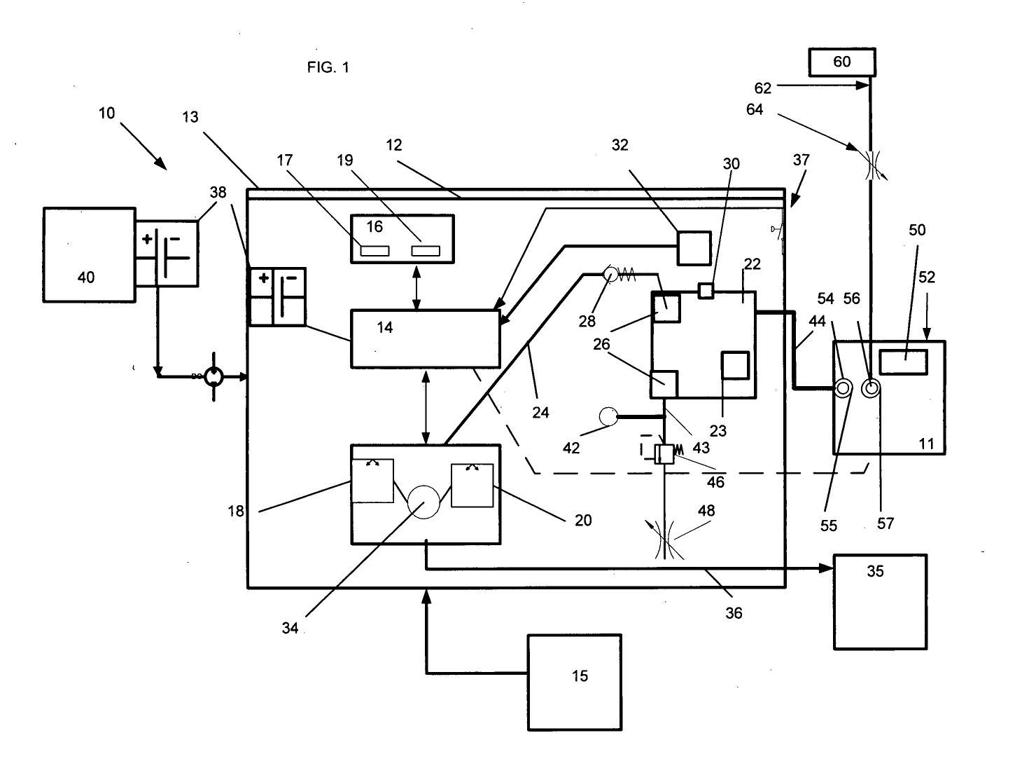

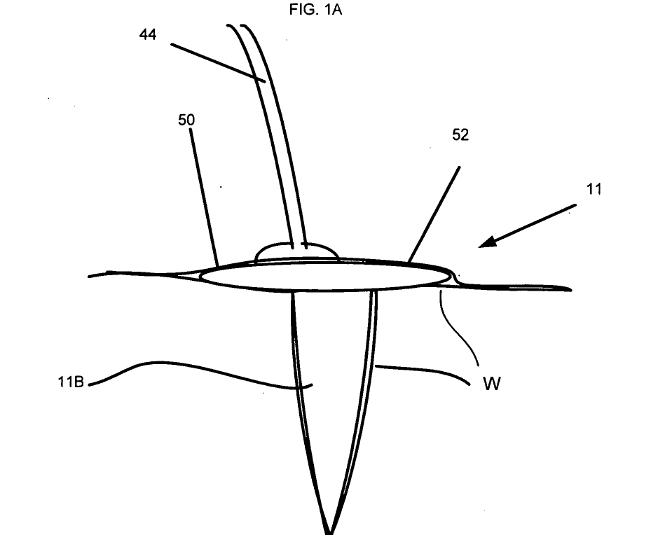

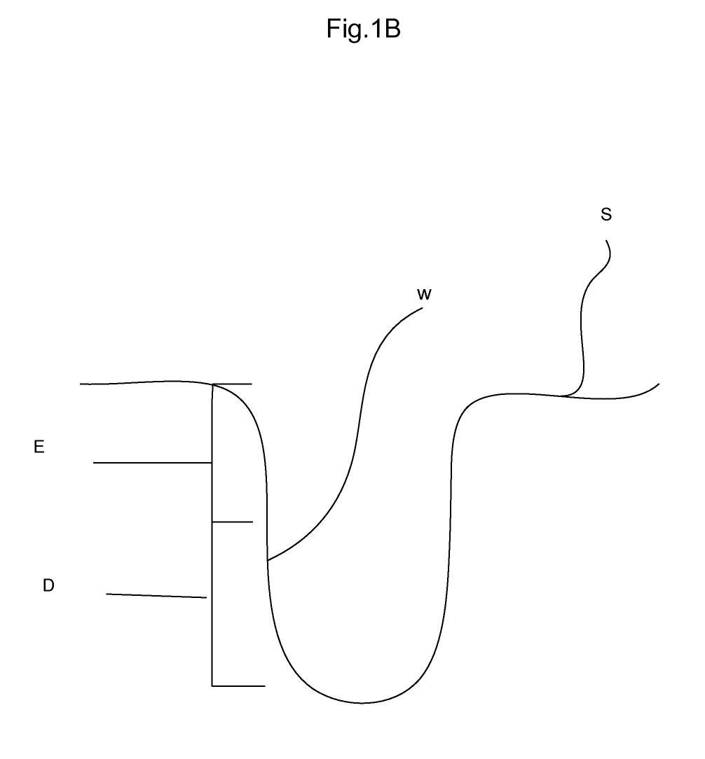

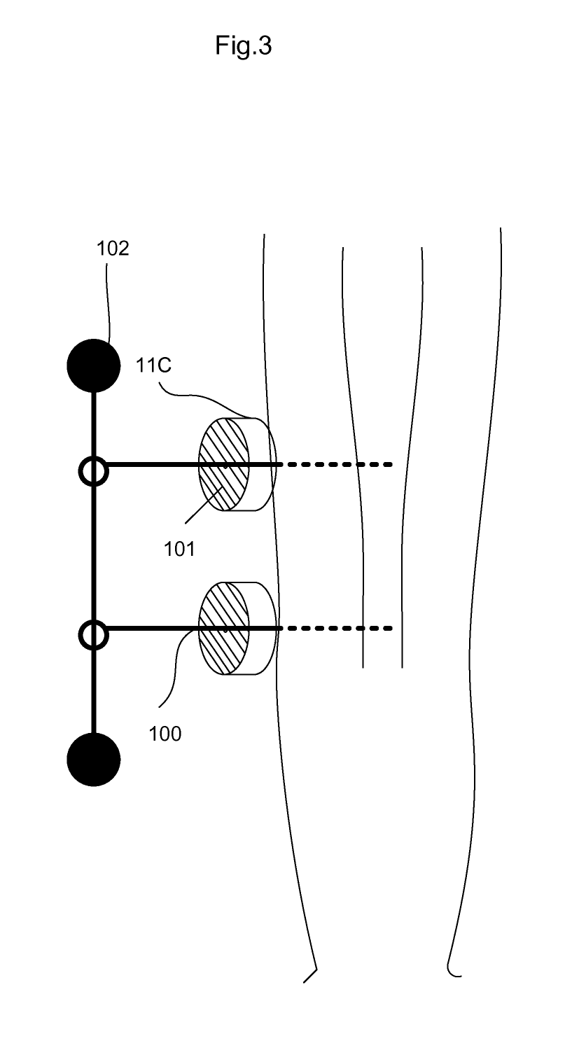

A wound W can extend well below the epidermis E of skin S and into the dermis D or hypodermis as depicted in FIGS. 1A and 1B. Wounds W may include tunnels, fistulas, wherein tunnels are abnormal tubular passages from a normal cavity or tube to a free surface or another cavity and fistulas are abnormal connections between an organ, vessel, or intestine and another structure or external fixation devices such as pins to hold bony structures in alignment as they heal as depicted in FIG. 3.

Grafts and fixation devices may be used in wounds to aid in repairing the wound W. Grafts include flaps of skin and other integumentary tissue from a donor to a recipient, or an autogenous graft of the patient's own tissue, inserted into a wound W to supply an absence or defect by attachment and growth into an integral part of the patient's tissues. As seen in FIG. 3, external fixation devices (EFD) such as pins 100 can extend outside the patient's skin into a rigid support structure 102 and penetrate skin S to immobilize a bony structure B while it heals. In these exemplary cases, the wound W is significantly deep and negative pressure therapy can preferably be employed to provide therapeutic treatment.

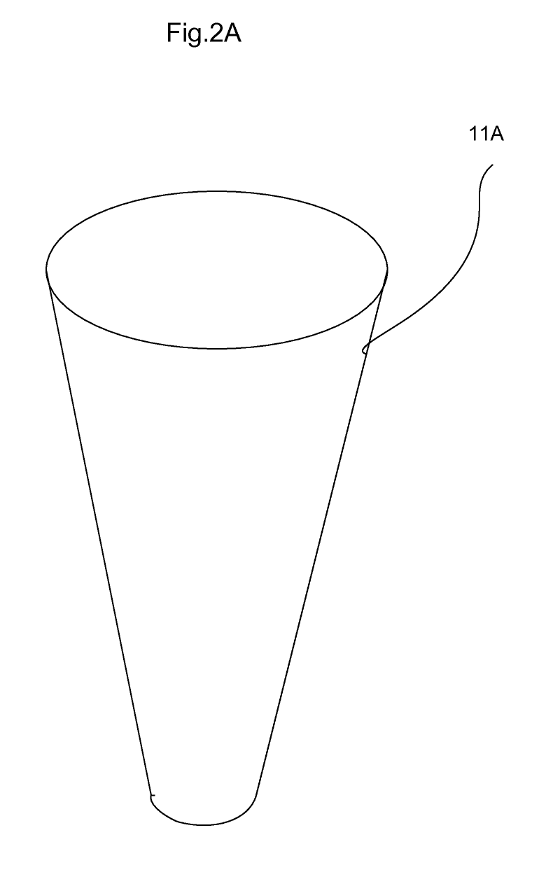

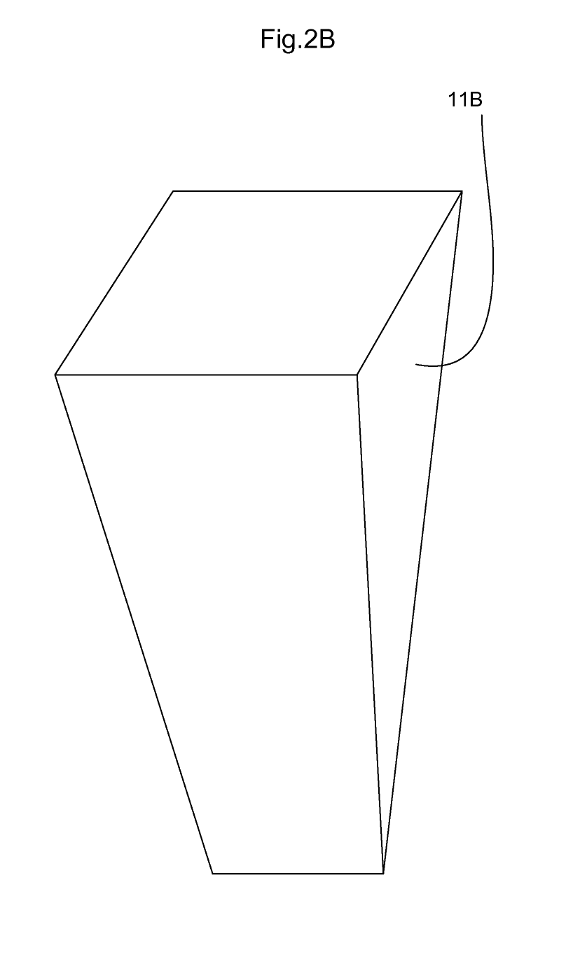

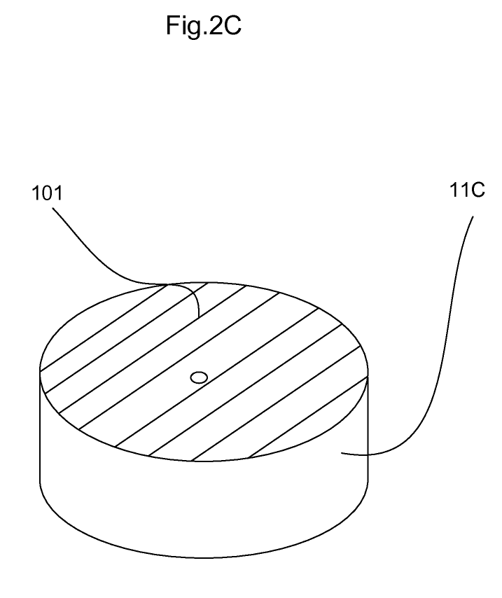

Specific physical configurations of the dressing 11 will preferably vary with the nature of the wound in order to provide a close fluidic association between the wound W and the compressible dressing 11. Specialized dressing shapes can be fashioned in various forms such as cylindrical, conical, pyramidal, wedges or disks which may be tailored to fit a wide variety of wounds. In this regard, exemplary parts 11A, 11B, and 11C may be fashioned as seen in FIGS. 2A, 2B and 2C.

The parts 11A, 11B can be formed for insertion into wound W and then covered with substrate 50, which can be an adjacent flat foam of similar porous material, and which extends over the part 11A or 11B and preferably extends to but not beyond margins surrounding the wound W. Another exemplary part 11C can be fashioned in a form of a disk having an aperture therein to receive pin 100 therethrough. The specific physical characteristics of the material for the dressings 11A, 11B, and 11C can be a hydrophilic, water-blown, open-cell, nonreticulated, polyurethane foam while substrate 50 can be a hydrophobic, reticulated, polyurethane-ether foam.

In this example, a side 101 of the disk 11C is provided with a hydrophilic hydrogel coating to form a seal about the aperture of the disk 11C with pin 100 and cover 52. The cover 52 encloses about the disk 11C and pin 11 and adheres to the patient's skin S and the hydrogel adhesive coating on side 101 provides a hydrophilic seal for gaps among the same.

An exemplary hydrogel formula for the adhesive coating on surface 101 of the circular foam disk 11C for the exterior fixator can contain the following ingredients by weight: 16-17% polypropylene glycol, 10-12% isophorone diisocyanate 7-9%, polyethylene oxide-based diamine in a 0.5-1.0% aqueous solution of NaCl. The hydrogel can be prepared by a process comprising the steps of:

1. Melting 9 g of diamine (Huntman Chemical), adding 7.5 g isophorone diisocyanate in water, and mixing together so the mixture remains liquid. (This is Part D.)

2. Mixing together 10.5 g glycol plus 52.7 g water plus 0.9 g NaCl. (This is Part C totaling 64.1 g, which is refrigerated to slow the final reaction.)

3. Refrigerating 6.8 g of polypropylene glycol to slow the final reaction. (This is Part B.)

4. Measuring 12.6 g of pre-polymer (diisocyanate). (This is Part A.)

5. Mixing B plus C plus D.

6. Mixing A with the mixture of B, C and D, stirring well, and pouring into a mold to form the desired shape of the coating for the foam dressing. Alternatively, the mixture can be poured directly onto the foam dressing and permitted to cure in place.

The formed hydrogel coating is applied to side 101 of the dressing disk 11C which can preferably be 1-3 inches in diameter and ⅜ to ⅝ inches thick. Aperture of disk 11C can be created in the center of the disk 11C and extend radially outward in the form of a slit throughout the thickness of the disk 11C. In this way, the disk 11C may be placed around the pin 101 of the external fixation device which is typically in place on the patient. The coated disk 11C may be used as a single dressing around the EFD pin 100.

One or more parts 11A, 11B, 11C or substrate 50 can be used together or independent from one another and in connection with membrane 52 such that the dressing 11 provides for fluid migration so the wound may be irrigated and the exudate collected in a canister 22 by a negative pressure wound therapy device as described herein. To this end as illustrated in FIG. 1, there is provided a negative pressure wound therapeutic device of the instant invention is generally designated by the numeral 10. The negative pressure wound therapeutic device 10 can preferably include a housing 12 which provides an improved therapeutic device with multiple uses and portability. The housing 12 can preferably be formed in a waterproof manner to protect components therein. In this regard, housing 12 can have a watertight sealed access panel 13 through which components can be accessed.

The device 10 can include a processor 14, which can be a microcontroller having an embedded microprocessor, Random Access Memory (RAM) and Flash Memory (FM). FM can preferably contain the programming instructions for a control algorithm. FM can preferably be non-volatile and retains its programming when the power is terminated. RAM can be utilized by the control algorithm for storing variables such as pressure measurements, alarm counts and the like, which the control algorithm uses while generating and maintaining the vacuum.

A membrane keypad and a light emitting diode LED or liquid crystal display (LCD) 16 can be electrically associated with processor 14 through a communication link, such as a cable. Keypad switches provide power control and are used to preset the desired pressure/vacuum levels. Light emitting diodes 17, 19 can be provided to indicate alarm conditions associated with canister fluid level, leaks of pressure in the dressing and canister, and power remaining in the power source.

Microcontroller 14 is electrically associated with, and controls the operation of, a first vacuum pump 18 and an optional second vacuum pump 20 through electrical connections. First vacuum pump 18 and optional second vacuum pump 20 can be one of many types including, for example, the pumps sold under the trademarks Hargraves® and Thomas®. Vacuum pumps 18 and 20 can use, for example, a reciprocating diaphragm or piston to create vacuum and can be typically powered by a direct current (DC) motor that can also optionally use a brushless commutator for increased reliability and longevity. Vacuum pumps 18 and 20 can be pneumatically associated with a disposable exudate collection canister 22 through a single-lumen tube 24.

In one embodiment, canister 22 has a volume which does not exceed 1000 ml. This can prevent accidental exsanguination of a patient in the event hemostasis has not yet been achieved at the wound site. Canister 22 can be of a custom design or one available off-the-shelf and sold under the trademark DeRoyal®.

In addition, a fluid barrier 26, which can be a back flow valve or filter, is associated with canister 22 and is configured to prevent fluids collected in canister 22 from escaping into tubing 24 and fouling the vacuum return path. Barrier 26 can be of a mechanical float design or may have one or more membranes of hydrophobic material such as those available under the trademark GoreTex™. Barrier 26 can also be fabricated from a porous polymer such as that which is available under the trademark MicroPore™. A secondary barrier 28 using a hydrophobic membrane or valve is inserted in-line with pneumatic tubing 24 to prevent fluid ingress into the system in the event barrier 26 fails to operate as intended. Pneumatic tubing 24 can connect to first vacuum pump 18 and optional second vacuum pump 20 through “T” connectors.

An identification member 30, such as radio frequency identification (RFID) tag, can be physically associated with the canister 22 and an RFID sensor 32 operably associated with the microcontroller 14 such that the microcontroller 14 can restrict use of the device 10 to a predetermined canister 22. Thus, if a canister 22 does not have a predetermined RFID chip, the device 10 will not operate. Another embodiment envisions software resident on microcontroller 14 which restricts the use of the device 10 to a predetermined time period such as 90 days for example. In this way, the patient using the device 10 may use the device 10 for a prescribed time period and then the device 10 automatically times out per a particular therapeutic plan for that patient. This also enables a reminder of the time and date for the next dressing change or physician appointment. It is also contemplated that the microcontroller 14 be operably provided with a remote control 15 and communication link, such as a transceiver, wherein the device 10 can be shut down remotely when a particular therapeutic plan for that patient has ended. Likewise, remote control 15 can be utilized to provide additional time after the therapeutic device times out.

Vacuum-pressure sensor 34 is pneumatically associated with first vacuum pump 18 and optional vacuum pump 20 and electrically associated with microcontroller 14. Pressure sensor 34 provides a vacuum-pressure signal to the microprocessor enabling a control algorithm to monitor vacuum pressure at the outlet of the vacuum pumps 18 and 20.

An acoustic muffler can be provided and pneumatically associated with the exhaust ports of vacuum pumps 18 and 20 and configured to reduce exhaust noise produced by the pumps during operation. In normal operation of device 10, first vacuum pump 18 can be used to generate the initial or “draw-down” vacuum while optional second vacuum pump 20 can be used to maintain a desired vacuum within the system compensating for any leaks or pressure fluctuations. Vacuum pump 20 can be smaller and quieter than vacuum pump 18 providing a means to maintain desired pressure without disturbing the patient. It is contemplated by the instant invention that pumps 18 and 20 can also be employed to create a positive pressure for purposes of applying pressure to an inflatable member 35, such as a cuff or pressure bandage, through tubing 36. A switch 37 can be operatively disposed on housing 12 in operable connection with microcontroller 14 to enable selection of positive and negative pressure from pumps 18/20.

One or more battery (ies) 38 can preferably be provided to permit portable operation of the device 10. Battery 38 can be Lithium Ion (LiIon), Nickel-Metal-Hydride (NiMH), Nickel-Cadmium, (NiCd) or their equivalent, and can be electrically associated with microcontroller 14 through electrical connections. Battery 38 can be of a rechargeable type which is preferably removably disposed in connection with the housing 12 and can be replaced with a secondary battery 38 when needed. A recharger 40 is provided to keep one battery 38 charged at all times. Additionally, it is contemplated that the device 10 can be equipped to be powered or charged by recharger 40 or by circuits related with microcontroller 14 if such source of power is available. When an external source of power is not available and the device 10 is to operate in a portable mode, battery 38 supplies power to the device 10. The battery 38 can be rechargeable or reprocessable and can preferably be removably stored in a waterproof manner within housing 12 which also likewise contains the pumps 18, 20 and microcontroller 14.

A second pressure sensor 42 is pneumatically associated with canister 22 through a sensor port 43. Pressure sensor 42 can be electrically associated with microcontroller 14 and provides a vacuum-pressure signal to microprocessor enabling control algorithm to monitor vacuum pressure inside canister 22 and dressing 11. A “T” connector can be connected to port 43, to pressure sensor 42 and a vacuum-pressure relief solenoid 46 configured to relieve pressure in the canister 22 and dressing 11 in the event of an alarm condition, or if power is turned off. Solenoid 46, can be, for example, one available under the trademark Parker Hannifin® or Pneutronics®; Solenoid 46 is electrically associated with, and controlled by, microprocessor of microcontroller 14. Solenoid 46 can be configured to vent vacuum pressure to atmosphere when an electrical coil associated therewith is de-energized as would be the case if the power is turned off. An orifice restrictor 48 may optionally be provided in-line with solenoid 46 and pneumatic tube 44 to regulate the rate at which vacuum is relieved to atmospheric pressure when solenoid 46 is de-energized. Orifice restrictor 48 is, for example, available under the trademark AirLogic®.

A wound dressing 11 in one embodiment can preferably include a sterile porous substrate 50, which can be a polyurethane foam, polyvinyl alcohol foam, gauze, felt or other suitable material, a semi-permeable adhesive cover 52 such as that sold under the trademark DeRoyal® or Avery Denison®, an inlet port 56 and a suction port 54. Additionally, the parts 11A, 11B, or 11C can be employed for various wound sites in combination or separate from substrate 50 which can be configured to distribute vacuum pressure evenly throughout the entire wound bed and has mechanical properties suitable for promoting the formation of granular tissue and approximating the wound margins.

When referring to dressing 11, this may include one or more of parts 11A, 11B, 11C and substrate 50. When vacuum is applied to dressing 11, there is created micro- and macro-strain at the cellular level of the wound stimulating the production of various growth factors and other cytokines, and promoting cell proliferation. Dressing 11 is fluidically associated with canister 22 through single-lumen tube 44. The vacuum pressure in a cavity formed within dressing 11 which is largely the same as the vacuum pressure inside canister 22 minus the weight of any standing fluid inside tubing 44.

A fluid vessel 60, which can be a standard IV bag, contains medicinal fluids such as aqueous topical antibiotics, analgesics, physiologic bleaches, or isotonic saline. Fluid vessel 60 is removably connected to dressing 11 though port 56 and single-lumen tube 62.

An optional flow control device 64 can be placed in-line with tubing 62 to permit accurate regulation of the fluid flow from vessel 60 to dressing 11. In normal operation, continuous wound site irrigation is provided as treatment fluids move from vessel 60 through dressing 11 and into collection canister 22. This continuous irrigation keeps the wound clean and helps to manage infection. In addition, effluent produced at the wound site and collected through the dressing 11 will be removed to canister 22 when the system is under vacuum. In the alternative, dressing 11 can be irrigated intermittently alternating between periods of continuous irrigation and vacuum suction only. Likewise, dressing 11 can be very effective when used with the above described system without any irrigation as a suction drainage device.

The device 10 is particularly well suited for providing therapeutic wound irrigation and vacuum drainage and provides for a self-contained plastic housing configured to be worn around the waist or carried in a pouch over the shoulder for patients who are ambulatory, and hung from the footboard or headboard of a bed for patients who are non-ambulatory. Membrane keypad and display 16 is provided to enable the adjustment of therapeutic parameters and to turn the unit on and off.

Depressing the power button on membrane switch 16 will turn the power to device 10 on/off. While it is contemplated that the membrane switch 16 be equipped with keys to adjust therapeutic pressure up and down, the microcontroller 14 can preferably be equipped to control the pressure in accordance with sensed pressure and condition to maintain pressure in an operable range between −70 mmHg and −150 mmHg with a working range of between 0 and −500 mmHg, for example. Although these pressure settings are provided by way of example, they are not intended to be limiting because other pressures can be utilized for wound-type specific applications. The membrane 16 can also be equipped with LED 17 to indicate a leak alarm and/or LED 19 indicates a full-canister alarm. When either alarm condition is detected, these LEDs will light in conjunction with an audible chime which is also included in the device 10. Alternatively, a Liquid Crystal Display (LCD) device can be readily substituted for indicators LED 17 and 19.

Housing 12 can incorporate a compartment configured in such a way as to receive and store a standard IV bag 60 or can be externally coupled to thereto. IV bag 60 may contain an aqueous topical wound treatment fluid that is utilized by the device 60 to provide continuous or intermittent irrigation. A belt clip can provided for attaching to a patient's belt and an optional waist strap or shoulder strap is provided for patients who do not or cannot wear belts.

Canister 22 is provided for exudate collection and can preferably be configured as currently known in the field with a vacuum-sealing means and associated fluid barrier 26, vacuum sensor port 43 and associated protective hydrophobic filter, contact-clear translucent body, clear graduated measurement window, locking means and tubing connection means. Collection canister 22 typically has a volume less than 1000 ml to prevent accidental exsanguination of a patient if hemostasis is not achieved in the wound. Fluid barriers 26 can be, for example, those sold under the trademark MicroPore® or GoreTex® and ensure the contents of canister 22 do not inadvertently ingress into pumps 18, 20 of housing 12 and subsequently cause contamination of thereof.

Pressure sensor 42 enables microcontroller 14 to measure the pressure within the canister 22 as a proxy for the therapeutic vacuum pressure under the dressing 11. Optionally, tubing 62 can be multilumen tubing providing one conduit for the irrigation fluid to travel to dressing 11 and another conduit for the vacuum drainage. Thus, IV bag 60, tubing 62, dressing 11 and canister 22 provide a closed fluid pathway. In this embodiment, canister 22 would be single-use disposable and may be filled with a solidifying agent 23 to enable the contents to solidify prior to disposal. Solidifying agents are available, for example, under the trademark DeRoyal® and Isolyzer®. The solidifying agents prevent fluid from sloshing around inside the canister particularly when the patient is mobile, such as would be the case if the patient were travelling in a motor vehicle. In addition, solidifying agents are available with antimicrobials that can destroy pathogens and help prevent aerosolization of bacteria.

At the termination of optional multilumen tubing 62, there can be provided a self-adhesive dressing connector 57 for attaching the tubing to drape 52 with substantially air-tight seal. Dressing connector 11 can have an annular pressure-sensitive adhesive ring with a release liner that is removed prior to application. Port 56 can be formed as a port cut in drape 52 and dressing connector 57 would be positioned in alignment with said port. This enables irrigation fluid to both enter and leave the dressing through a single port. In an alternative embodiment, tube 62 can bifurcate at the terminus and connect to two dressing connectors 57 which allow the irrigation port to be physically separated from the vacuum drainage port thus forcing irrigation fluid to flow though the entire length of the dressing if it is so desired. Similarly, port 54 and connector 55 can be provided to connect optional multilumen tubing 44 to dressing 11. In this arrangement, the second lumen may be used to directly measure the pressure in dressing 11.

Fluid vessel 60 can be of the type which includes a self-sealing needle port situated on the superior aspect of the vessel 60 and a regulated drip port situated on the inferior aspect of the vessel. The needle port permits the introduction of a hypodermic needle for the administration of aqueous topical wound treatment fluids. These aqueous topical fluids can include a topical anesthetic such as Lidocaine, antibiotics such as Bacitracin or Sulfamide-Acetate; physiologic bleach such as Chlorpactin or Dakins solution; and antiseptics such as Lavasept or Octenisept. Regulated drip port permits fluid within vessel 60 to egress slowly and continuously into porous substrate 50 whereupon the therapeutic benefits can be imparted to the wound site. Single-lumen drainage tube 44 provides enough vacuum to keep the dressing 11 at sub-atmospheric pressure and to remove fluids, which include the irrigation fluid and wound exudates. With this modification, the need for an external fluid vessel and associated tubing and connectors can be eliminated making the dressing more user friendly for patient and clinician alike.

In the case of deep wound W, one method for providing negative pressure wound healing in a deep wound having a cavity includes the steps of fashioning at least a first part of a therapeutic dressing 11A, for example, to fit generally within the cavity of wound W. The negative pressure wound therapeutic device 10 is operably associated with the therapeutic dressing 11 for enabling compressing and/or transferring fluid across at least part of the therapeutic dressing 11 and enabling the pump in a manner to deliver therapy to the patient.

The therapeutic dressing 11 can include a first polyurethane foam part 11A which is cut to generally fit within the cavity of wound W and substrate 50 can cover the part 11A and extend to but not beyond the margins of the wound cavity. Semi-permeable membrane 52 is wrapped about the part 11A and 50 and in generally sealed contact with skin S about the wound W.

The negative pressure therapeutic device 10 can be enabled for a period to foster at least partial closure of the wound W rendering a least a partially closed cavity and cause at least partial migration the part 11A from the wound W. Further steps can include removing and reducing the size of part 11A to provide a therapeutic dressing 11 having a first part 11A′ with reduced size relative the part 11A to fit generally within the at least partially closed cavity. Similarly, negative pressure wound therapeutic device 10 is operably associated with the therapeutic dressing 11 for enabling one of compressing the therapeutic dressing and transferring fluid across at least part of the therapeutic dressing in a manner to deliver therapy to the patient.

The negative pressure wound therapeutic device 10 can be enabled again for a period to foster closure of the wound rendering a closure and cause migration the first part 11A′ from the cavity of the wound W and the part 11A′ removed. Optionally, if further healing about the site is required, the dressing 11 may employ substrate 50 over the closed cavity and enable negative pressure wound therapeutic device 10 with the therapeutic dressing 11 to deliver further therapy to the patient.

The method of use of the deep wound dressings can include cutting to form parts 11A′ to approximate the part surfaces with the wound surfaces. Applying negative pressure therapy to the wound can be for a 24-48 hour period prior to resizing parts and dressing 11 which are changed every 24-48 hours during the healing process. The dressing size will vary as wound margins are reduced while re-epithelialization and revascularization proceed with growth of granular tissue. Thus all surfaces of the deep wound site are irrigated and drained throughout the healing process.

Another embodiment shows the employment of part 11C which is disposed about the pin 100 and adjacent skin S and wound W. Aside from the different part 11C, the other methodology described above can be employed.

In this regard, semi-permeable drape 52 is attached and sealed over the dressing parts 11A, 11B, 11C and/or 50 and periwound. A hole approximately ⅜″ diameter can be made in drape 52 central to porous substrate 50. Fluid vessel 60 is attached by adhesive annular ring 57 with port 56 aligned with the hole previously cut in drape 52. Once the fluid vessel 60 is hermitically sealed to the drape 52, a properly prepared hypodermic needle is inserted in self-sealing needle port and fluid vessel 60 subsequently filled with the desired aqueous topical wound treatment solution.

For the majority of applications, the technique for providing therapeutic wound irrigation and vacuum drainage is illustrated. The single lumen drainage tube 44 is provided for the application of vacuum and removal of fluids from the wound site. Fluid vessel 60 can be situated outside and superior to semi-permeable substrate 50. An annular adhesive ring 57 is provided on port 56 for attachment of single-lumen irrigation tubing 62 to drape 52. Similarly, a needle port permits the introduction of a hypodermic needle for the administration of aqueous topical wound treatment fluids as described above, for example, a caregiver may want to add a topical antibiotic to a bag of isotonic saline. Adjustable optional flow control device 64 permits fluid within vessel 60 to egress slowly and continuously into porous substrate 50 through hole 56 in drape 52 whereupon the therapeutic benefits can be imparted to the wound site. Single-lumen drainage tube 44 provides enough vacuum to keep the dressing 11 at sub-atmospheric pressure and to remove fluids which include the irrigation fluid and wound exudates.

What is claimed is:

1. A method for providing negative pressure wound healing in a deep wound having a cavity, which includes the steps of:
(a) fashioning at least a first part of a therapeutic dressing to fit generally within the cavity;
(b) operably associating a fluid moving means with the therapeutic dressing for enabling one of compressing the therapeutic dressing and transferring fluid across at least part of the therapeutic dressing; and
(c) enabling the fluid moving means in a manner to deliver therapy to the patient.

2. The method for providing negative pressure wound healing of claim 1, wherein therapeutic dressing in step (a) is further characterized to include a first foam portion which is cut to generally fit within the cavity and a second foam portion extends outside the wound cavity extending to but not beyond the margins of the wound.

3. The method for providing negative pressure wound healing of claim 2, wherein therapeutic dressing in step (a) is further characterized to include a semi-permeable membrane wrapped about the foam portions and in generally sealed contact with skin about the wound.

4. The method for providing negative pressure wound healing of claim 1, which further characterizes the step (c) to enable the fluid moving means for a period of time to foster at least partial closure of the wound rendering a least a partially closed cavity and cause at least partial migration the first part of the therapeutic dressing from the wound cavity and further includes the steps of:
(d) removing at least part of the first part of the therapeutic dressing;
(e) providing a therapeutic dressing having a first part with reduced size relative the first part of the therapeutic dressing in step (a) to fit generally within the at least partially closed cavity;
(f) operably associating a fluid moving means with the therapeutic dressing with reduced size for enabling one of compressing the therapeutic dressing and transferring fluid across at least part of the therapeutic dressing; and
(g) enabling the fluid moving means in a manner to deliver therapy to the patient.

5. The method for providing negative pressure wound healing of claim 4, which further characterizes the step (g) to enable the fluid moving means for a period of time to foster closure of the wound rendering a closure of the cavity and cause migration the first part of the therapeutic dressing from the wound cavity and further includes the steps of:
(h) removing the first part of the therapeutic dressing;
(i) providing a therapeutic dressing having a second compressible part over the closed cavity;
(j) operably associating a fluid moving means with the therapeutic dressing for enabling one of compressing the therapeutic dressing and transferring fluid from across at least part of the therapeutic dressing; and
(k) enabling the fluid moving means in a manner to deliver therapy to the patient.

6. The method for providing negative pressure wound healing of claim 4, which further characterizes the step (g) to enable the fluid moving means for a period of time to foster closure of the wound rendering a closure of the cavity and cause migration the first part of the therapeutic dressing.

7. The method for providing negative pressure wound healing of claim 1, wherein the therapeutic dressing includes at least one foam member.

8. The method for providing negative pressure wound healing of claim 1, wherein the therapeutic dressing includes at least two foam portions, wherein one of the portions is configured fit generally within the cavity and the other portion is configured to be disposed outside the cavity extending to but not beyond the margins of the wound.

9. A method for providing negative pressure wound healing in a wound having a cavity in which part of an external fixation device extends, which includes the steps of:
(a) fashioning at least a first part of a therapeutic dressing to fit generally about the part of the external fixation device adjacent the cavity of the wound;
(b) operably associating a fluid moving means with the therapeutic dressing for enabling one of compressing the therapeutic dressing and transferring fluid across at least part of the therapeutic dressing; and
(c) enabling the fluid moving means in a manner to deliver therapy to the patient.

10. The method for providing negative pressure wound healing of claim 9, wherein the first part is further characterized to include foam.

11. The method for providing negative pressure wound healing of claim 10, wherein a side of the foam is coated with a hydrogel.

12. The method for providing negative pressure wound healing of claim 10, wherein the foam is polyurethane foam.

13. The method for providing negative pressure wound healing of claim 9, wherein therapeutic dressing in step (a) is further characterized to include a semi-permeable membrane wrapped about the first part and in generally sealed contact with skin about the wound.

14. The method for providing negative pressure wound healing of claim 9, wherein therapeutic dressing in step (a) is further characterized such that the part of the external fixation device is a pin and the first part of the therapeutic device includes an aperture to receive the pin.

15. The method for providing negative pressure wound healing of claim 9, which further characterizes the step (c) to enable the fluid moving means for a period of time to foster at least partial healing and further includes the steps of:
(d) removing the first part of the therapeutic dressing;
(e) providing a therapeutic dressing having a second part formed to fit generally about a part of the external fixation device adjacent the cavity of the wound;
(f) operably associating a fluid moving means with the therapeutic dressing for enabling one of compressing the therapeutic dressing and transferring fluid across at least part of the therapeutic dressing; and
(g) enabling the fluid moving means in a manner to deliver therapy to the patient.

16. A therapeutic member for use in negative pressure wound healing, which includes a porous member formed and includes a hydrogel coating on a side thereof.

17. The therapeutic member for use in negative pressure wound healing of claim 15 wherein the porous member includes an aperture configured to fit generally about a part of the external fixation device.

18. The therapeutic member for use in negative pressure wound healing of claim 16, wherein the hydrogel includes polypropylene glycol, isophorone diisocyanate, and polyethylene oxide-based diamine in an aqueous solution of NaCl.

19. The therapeutic member for use in negative pressure wound healing of claim 18, wherein the hydrogel includes about by weight: 10-20% polypropylene glycol, 5-15% isophorone diisocyanate 5-15%, polyethylene oxide-based diamine in a 0.1-3.0% aqueous solution of NaCl.

20. The therapeutic member for use in negative pressure wound healing of claim 18, wherein the hydrogel includes about by weight: 16-17% polypropylene glycol, 10-12% isophorone diisocyanate 7-9%, polyethylene oxide-based diamine in a 0.5-1.0% aqueous solution of NaCl.

21. A hydrogel for use in negative pressure wound healing prepared by a process comprising the steps of:
(a) melting about 9 g of diamine and adding about 7.5 g isophorone diisocyanate in water and mixing together with the first part such that the mixture remains liquid to form a first part;
(b) mixing together about 10.5 g glycol with about 52.7 g water and 0.9 g NaCl to form a second part;
(c) cooling the third part to slow the final reaction.
(d) cooling about 6.8 g of polypropylene glycol to form a third part;
(e) measuring about 12.6 g of pre-polymer (diisocyanate) to form a fourth part;
(f) mixing the first, second, third parts; and
(g) mixing the fourth part with the mixture of step (f) to form a final mixture.

22. The hydrogel for use in negative pressure wound healing prepared by a process of claim 21, which includes the step of pouring the final mixture into a mold to form a hydrogel of a predetermined configuration.

23. The hydrogel for use in negative pressure wound healing prepared by a process of claim 22, wherein the formed hydrogel is applied to a side of a foam part.

24. The hydrogel for use in negative pressure wound healing prepared by a process of claim 21, which includes the step of pouring the final mixture directly onto a side of foam part surface and subsequently allowing it to cure thereon.
